(19) United States
(12) Patent Application Publication
(10) Pub. No.: US 2004/0153007 A1
(43) Pub. Date: Aug. 5, 2004

(54) Physiological monitoring and system

(76) Inventors: Shawn Harris, Santa Monica, CA (US); Seth Joyner, Paradise Valley, AZ (US)

Correspondence Address: GLENN C. BROWN, PC, 777 NW WALL STREET, SUITE 308, BEND, OR, 97701, US

(21) Appl. No.: 10/753,794

(22) Filed: Jan. 8, 2004

Related U.S. Application Data
(63) Continuation of application No. PCT/US02/19713, filed on Jun. 19, 2002.
(60) Provisional application No. 60/299,602, filed on Jun. 19, 2001.

(30) Foreign Application Priority Data
Dec. 27, 2002 (WO) 02/102240 A2

(51) Int. Cl. A61B 5/103; A61B 5/00

(52) U.S. Cl. 600/587; 600/300

(57) ABSTRACT

A physiological monitoring system that collects real time physiological data from one or more users and wirelessly transmits the data to a remote data base. The remote data base is accessible to utilize the stored data to assess the athlete's performance. The system can also include position sensors to determine and transmit an athlete's precise location on a playing field or court.

### CROSS REFERENCE TO RELATED APPLICATION

The present application is a continuation of PCT/US02/19713, filed on Jun. 19, 2002, which published as WO 02/102240 A2on Dec. 27, 2002, which is a continuation in part of U.S. provisional patent application Serial No. 60/299,602, filed on Jun. 19, 2001. The priority of the prior application is expressly claimed and its disclosure is hereby incorporated by reference in its entirety.

### BACKGROUND OF THE INVENTION

In recent years professional and amateur sports have begun to exploit the power of technological advancements to enhance athlete's performance and better their entities. The majority of progress made, to date, has been focused on providing athletes with advanced supplements and diet regiments. While the improvements made are incredibly beneficial, there remains greater opportunity to further enrich the training structure by providing athletes with advanced training tools based on real-time physiological data.

Current methods of physiological data monitoring of athletes require periodic testing or monitoring of the athlete's functions such as temperature and respiration. While useful, such data do not provide the precise temporal information that would most useful in assessing an athlete's conditioning or performance.

### SUMMARY OF THE INVENTION

This invention includes a system that collects physiological and/or location information from one or multiple athletes, and maintains a data base of information specific to that athlete.

In one embodiment the system includes a shoe with embedded sensors that measure force, stride length, foot strike, stride frequency, and speed. The athlete also wears electrodes, such as wireless Surface Electromyography button electrodes, to measure physiological functions such as pulse, heart rate, blood oxygen saturation, skin temperature, and the temporal patterns of selected muscle activity. The data acquired by the sensors are transmitted to a microprocessor that in one preferred embodiment is also located in the shoe, or which is worn in another location by the user. The microprocessor includes a transmitter, and is in wireless communication with a data receiver or alternatively, one or more antennae that are located around the playing field or court. The collected data is transmitted to the antennae, and from the antennae to a host server using a standard protocol such as Bluetooth®. In addition to receiving the physiological data, the antennae are adapted to determine the relative angular location of the transmitter with respect to each player. The host server receives this location information and determines the location of each player. The host server is programmed to process the data received for each player, and transmits the data to a data base that includes files for each athlete.

The data base is automatically populated with each athlete's physiological and location data received from the host server. In preferred embodiments the data is encrypted for security and privacy. In a preferred embodiment the data base is accessible via an internet connection, and is available real time for use in assessing the physical performance of the athlete, and the performance of multiple athletes. For example, during a game or a practice a coach could access the data base immediately after a particular play, and evaluate players' performance and positioning on the field or court during the play.

The host server can also include removable storage to be used instead of the real time wireless capability. This permits athletes out of range of a wireless access point to use the system. This will be useful for runners, bikers, and others who may be training out of range of an access point. In this scenario when the training session is done the user then downloads the data to a PC and then thru the Internet to the database.

These and other aspects of the invention will now be described by reference to the drawings.

### BRIEF DESCRIPTION OF THE DRAWINGS

FIG. 1 is a schematic diagram of a system according to the invention.

FIG. 2 is a schematic cross-sectional view of a shoe according to the invention, and showing schematically the sensor plate and microprocessor/controller.

FIG. 3 is a perspective view of players on a court and wearing sensors according to a preferred embodiment of the invention.

### DETAILED DESCRIPTION OF THE PREFERRED EMBODIMENT

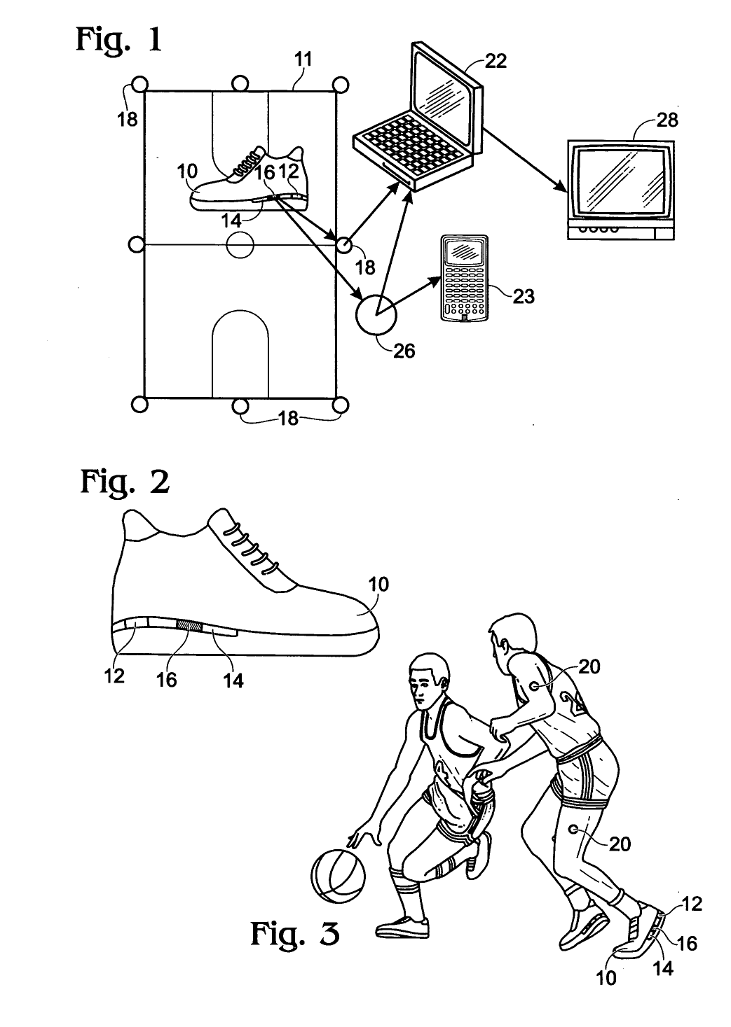

Referring now to FIGS. 1-3, one preferred embodiment of the invention includes a system that collects physiological information from one or multiple athletes, and maintains a data base of information specific to that athlete. The information in the data base can be accessed real time, uses a wireless network to connect components including physiological sensors, a monitor, a database and user interfaces. These components wirelessly deliver real time location and/or physiological data to the database for the use of athletes and trainers. The system can be used to provide analysis and guidance to the athlete and trainer based on goals and performance measured against goals.

In one embodiment shown schematically in FIG. 1, the system includes a shoe 10 with embedded sensors 12 that measure force, stride length, foot strike, stride frequency, and/or speed. The sensors are preferably mounted or embedded in a rigid or semi rigid plate 14 that underlies the footed of the shoe. The sensors are in communication with a microprocessor/controller 16 that receives the data, converts analog signals to digital signals, and transmits the digital signals to one or more receivers or antennae 18. In other preferred embodiments, the data can be temporarily stored by the microprocessor/controller, or on another memory device carried or worn by the user, or by the receiver 18. This embodiment is particularly useful for athletes such as long distance runners or skiers whose activities would take them beyond the range of low power transmitters or antennae. In one embodiment the receiver includes a plurality of antennae located around the playing field or court. In an alternate embodiment, the receiver can be laptop computer, PDA, or 3G cell phone. The transmission of data is preferably accomplished using Bluetooth® protocol.

In addition to or instead of the shoe embedded sensors, the athlete can wear electrodes 20 (FIG. 2), such as wireless Surface Electromyography button electrodes, taped to their skin to measure physiological functions such as pulse, heart rate, blood oxygen saturation, skin temperature, and the temporal patterns of selected muscle activity. Such sensors can be used to measure and monitor limb movements and motion sequences. The data acquired by the sensors are wirelessly transmitted to the microprocessor 16, which is also located in the shoe, or which is worn in another location by the user. The physiological data received from these sensors is processed as described above with respect to the shoe-mounted sensors.

In one preferred embodiment that is particularly suited to team sports, the microprocessor/controller 16 is in wireless communication with one or more directional antennae 18 (FIG. 1) that are located around the playing field or court. By directional antennae we mean antennae that can determine the angular position of the transmitter relative to the antenna. The collected data is transmitted by the microprocessor to the antennae using a standard protocol such as Bluetooth®, although the invention is not intended to be so limited. The data received by the antennae are transmitted to a host server. As mentioned above, in one preferred embodiment the antennae are adapted to determine the relative angular location of the antennae with respect to each player. The host server receives this location information and by methods similar to those principles utilized in global positioning systems, determines the location of each player on the field or court. This temporal location information can also be transmitted to the data base for real time or delayed viewing.

The host server 22 is programmed to process the data received for each player, and transmits the data to a data base that includes files for each athlete. In the instance of location information, a file can also be created for a pre-selected group of players of interest in a particular scenario, e.g. the offensive or defensive line of a football team. The data base is automatically populated with each athlete's or group's physiological and location data when received from the host server. In preferred embodiments the data is encrypted for security and privacy.

In a preferred embodiment the data base is maintained on a remote site and is accessible via an internet connection, and is available real time for use in assessing the physical performance of the athlete or a desired group of athletes. For example, during a game or a practice a coach could access the data base immediately after a particular play, and evaluate players' performance and positioning on the field or court during the play. As another example, each player's physiological functions can be monitored through the course of the game or activity to monitor fatigue.

The microprocessor/controller or host server can also include memory for data storage during the athlete's performance. This feature permits athletes out of range of an antennae or receiving device to collect the data and later transmit the data to the data base. This will be useful for runners, bikers, and others who may be training out of range of an access point. In this scenario when the training session is done the user then downloads the data to a PC, 3G cellular telephone, or PDA, and then through the internet to the database.

Once in the data base, the data for each athlete can be accessed and utilized according to the nature of the activity. An individual athlete can utilize the data base to track progress during training, or to evaluate individual performance parameters such as heart rate or split times. Teams can use the information to identify flaws in the team's execution of plays, track the physical conditioning of its athletes during play or during the off-season from remote locations. In another interesting aspect of the invention, the player location information can be utilized by broadcast media to reconstruct plays during broadcast of a game, and to highlight key plays by either team or individual athletes during the game. The invention might also have applications in the regulation of play to determine a player's location on the field at a specific time.

While these and other features of the invention have been described with respect to the preferred embodiments described above, those of skill in the art will recognize that numerous modifications in detail and arrangement of the invention are possible without departing from the scope of the following claims.

What is claimed is:

1. A physiological monitoring system comprising:
at least one physiological sensor in communication with a user and adapted to detect a physiological function of the user and to generate a physiological data signal responsive thereto;
a monitor in communication with the at least one sensor and adapted to receive the physiological data signal therefrom and to transmit the physiological data signal;
a data receiver/transmitter in communication with the monitor; and,
a data base in communication with the data receiver/transmitter.

2. A physiological monitoring system according to claim 1 further comprising the monitor in wireless communication with the data receiver/transmitter.

3. A physiological monitoring system according to claim 1 further comprising the at least one sensor in wireless communication with the monitor.

4. A physiological monitoring system according to claim 1 further comprising the data receiver/transmitter in communication with a remote data base.

5. A physiological monitoring system according to claim 4 further comprising the data receiver/transmitter in wireless communication with the database via the internet.

6. A physiological monitoring system according to claim 1 further comprising an analog to digital converter adapted to convert an analog physiological signal to a digital signal.

7. A physiological monitoring system according to claim 1 wherein the database includes files for a plurality of users.

8. A physiological monitoring system according to claim 1 wherein the at least one sensor detects at least one physiological function selected from the group consisting of heart rate, respiration rate, blood oxygen level, muscle activity, skin temperature, body temperature, and skin temperature.

9. A physiological monitoring system according to claim 1 wherein the monitor includes a memory for storing the at least one physiological data signal received from the at least one sensor.

10. A physiological monitoring system according to claim 1 wherein the monitor is located in the shoe of a user.

11. A physiological monitoring system according to claim 1 wherein the monitor is worn by the user.

12. A physiological monitoring system according to claim 1 wherein the data in the database can be accessed real-time.

13. A physiological monitoring system according to claim 1 wherein the database can be accessed from a location remote from the user and the data receiver/transmitter.

14. A system for determining the position of at least one user comprising:
a first receiver proximate to a first user;
at least one transmitter in communication with the first receiver;
a signal processor in communication with the first receiver and adapted to generate a first position defining signal specifying the location of the first user relative to the at least one transmitter;
a data receiver/transmitter in communication with the signal processor and adapted to receive the first position defining signal from the signal processor;
a data base in communication with the data receiver/transmitter.
